(19) United States
(12) Patent Application Publication
(10) Pub. No.: US 2006/0074231 A1
(43) Pub. Date: Apr. 6, 2006

(54) Amino acid-containing compounds and derivatives labeled with halides and method of making

(75) Inventors: Tiberiu Mircea Siclovan, Rexford, NY (US); Faisal Ahmed Syud, Clifton Park, NY (US); Bruce Fletcher Johnson, Scotia, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/957,220

(22) Filed: Oct. 4, 2004

(51) Int. Cl. C07K 14/00; C07D 209/18; C07H 21/04; C07C 227/16

(52) U.S. Cl. 530/409; 548/495; 548/530; 536/25.32; 562/553

(57) ABSTRACT

A method of labeling amino acid-containing compounds and derivatives thereof with a halide moiety comprises reacting a nucleophilic moiety on such compounds with a halogenated electrophilic compound. Radioactive halide-labeled amino acid-containing compounds can be targeted to diseased sites and provide a means to diagnose and/or treat the disease.

### BACKGROUND OF THE INVENTION

The present invention relates to amino acid-containing compounds and their derivatives labeled with one or more halogen atoms and to methods of making such labeled amino acid-containing compounds and derivatives.

The growing need for the early diagnosis and assessment and/or treatment of diseases can potentially be addressed by pharmaceuticals that preferentially accumulate at the disease sites. In diagnostic applications, these pharmaceuticals can elucidate the state of the disease through its distinctive biology expressed as disease markers that are not present, or are present in diminished levels, in healthy tissues. In therapeutic applications, these pharmaceuticals can deliver an enhanced dose of therapeutic agents to the disease sites through specific interactions with the disease markers. By specifically targeting physiological or cellular functions that are present only in disease states, these pharmaceuticals can report exclusively on the scope and progress of that disease or exclusively target the diseased tissue. A signal-generating moiety is a key element of these diagnostic pharmaceuticals, which produce differentiated signals at the disease sites.

In certain situations, these pharmaceuticals are based on peptides or derivatives thereof that bind specifically to disease markers. The peptides or derivatives thereof are labeled with moieties that generate a signal that can be detected by imaging equipment for the purposes of disease diagnosis. Alternatively, the moieties can comprise a radioisotope for the purposes of disease therapy.

Positron emission tomography (“PET”) has gained acceptance as a technique for diagnostic imaging because of its capability of providing images with high resolution in addition to its non-invasive nature. In PET, gamma photons having 511 keV energy produced during positron annihilation decay are detected. In the clinical setting, fluorine-18 (F-18) is one of the most widely used positron-emitting nuclides. However, its relatively short half life of 110 minutes has limited or precluded its use with constructs (such as antibodies, antibody fragments, or receptor-targeted peptides) that would require relatively long time to accumulate sufficiently at the target. Furthermore, the relatively short half life of F-18 would necessitate the manufacture of the F-18-labeled pharmaceutical immediately before its use and a short time required for such manufacture. However, these requirements are inconsistent with the currently known complicated chemistry that is required to link inorganic fluoride species to such organic targeting vectors.

Therefore, a continued need exists for a rapid method of labeling peptides and their derivatives with short-lived radioisotopes. In particular, it is very desirable to provide a rapid method for labeling peptides and their derivatives with short-lived halogen radioisotopes.

### SUMMARY OF THE INVENTION

The present invention provides a method of labeling an amino acid-containing compound or a derivative thereof with a halide moiety. The method comprises reacting a halogenated electrophilic compound with a nucleophilic moiety of the amino acid-containing compound or a derivative thereof.

In one aspect of the present invention, the halide moiety in the halogenated electrophilic compound is a radioactive halide.

In another aspect of the present invention, the radioactive halide is selected from the group consisting of halide radioisotopes that emit positrons.

In still another aspect, the amino acid-containing compound is a peptide or a peptide derivative, and the nucleophilic moiety is conjugated to a residue of the peptide or peptide derivative.

In still another aspect, the amino-acid containing compound comprises at least one amino-acid residue and at least a residue of at least another type of monomeric units in the backbone chain, wherein the nucleophilic moiety is conjugated to the at least one amino-acid residue.

In still another aspect, the present invention provides a pharmaceutical labeled with a radioactive halide moiety, wherein the pharmaceutical comprises a peptide or a peptide derivative.

In still another aspect, the present invention provides a set of separate compounds comprising a first compound comprising a peptide or a peptide derivative that comprises a nucleophilic moiety, and a second compound that is electrophilic and comprises a halide moiety. The compounds readily react with one another to produce a halide-labeled peptide or peptide derivative.

Other features and advantages of the present invention will be apparent from a perusal of the following detailed description of the invention and the accompanying drawings in which the same numerals refer to like elements.

### BRIEF DESCRIPTION OF THE DRAWINGS

FIG. 1 shows the MALDI-TOF (_{matrix assisted laser desorption ionisation time-of-flight)} mass spectrum of J₁FLGFL-NH₂, wherein J₁ is 3,4,5-trimethoxybenzyl glycine, and wherein F, L, and G conventionally denote phenylalanine, leucine, and glycine, respectively.

FIG. 2 shows the MALDI-TOF mass spectrum of J₂FLGFL-NH₂, wherein J₂ is 3-(3,4,5-trimethoxyphenyl)propionic acid.

### DETAILED DESCRIPTION OF THE INVENTION

The present invention provides a method of labeling an amino acid-containing compound or a derivative thereof with a halide moiety. The method comprises reacting a halogenated electrophilic compound with a nucleophilic moiety of the amino acid-containing compound or derivative thereof.

In one aspect of the present invention, the halide moiety in the halogenated electrophilic compound is a radioactive halide. The radioactive halide-labeled amino acid-containing compounds or derivatives thereof disclosed in the present invention are useful in diagnostic and/or therapeutic applications. For example, the amino acid-containing compound or derivative thereof can preferentially accumulate at a disease site by preferentially or specifically binding to an epitope expressed on the surface of cells of the diseased tissue. Alternatively, the amino acid-containing compound or a derivative thereof can preferentially accumulate at a disease site by binding to an enzyme overproduced by the diseased tissue. Thus, the radioactive halide-labeled amino acid-containing compound carries the radioactive halide label with it to the disease site, which can then be imaged by detecting and measuring the differentiated level of radioactivity. Alternatively, the radioactive halide-labeled amino acid-containing compound or a derivative thereof can have a therapeutic effect when the emitted radiation can kill the surrounding diseased tissue or otherwise stop its growth.

Suitable for halogen radioisotopes for labeling a amino acid-containing compound or derivative thereof in the present invention to produce a diagnostic or therapeutic pharmaceutical are fluorine-18, iodine-120, iodine-123, iodine-124, iodine-125, iodine-126, iodine-131, iodine-133, bromine-75, bromine-76, bromine-77, bromine-78, chlorine-34, chlorine-38, and chlorine-39. It should be noted that any of these halogen isotopes is present in the form of a combined halide in a compound with an electrophilic moiety.

Isotopes preferred for imaging applications include: fluorine-18, iodine-123, iodine-125, iodine-131, bromine-75, bromine-76, and bromine-77.

In another aspect of the present invention, the radioactive halide is selected from the group consisting of halide radioisotopes that emit positrons. A preferred radioisotope for PET is fluorine-18.

The nucleophilic moiety of the amino acid-containing compound or derivative thereof of the present invention is preferably attached to the amino acid-containing compound or derivative thereof by a direct covalent bond or a linkage selected from the group consisting of divalent saturated or unsaturated hydrocarbyl groups, and derivatives thereof. In one embodiment, the divalent saturated or unsaturated hydrocarbyl group, or a derivative thereof, is a chain having from one to ten carbon atoms, preferably from one to six carbon atoms, inclusive. In another embodiment, the linkage can be —(O—CH₂—CH₂)ₙ—, —(S—CH₂—CH₂)ₙ—, or —(NR¹—CH₂—CH₂)ₙ—, wherein n is an integer such that 1≦n≦5, preferably 1≦n≦3, and R¹ is —CₘHₘ₊₁, and m is an integer such that 1≦m≦5, preferably 1≦m≦3. It should be understood that n and m are independently selected.

The nucleophilic moiety comprises from one to three aromatic rings, substituted with one or more electron-donating groups, such as —OR¹, or —SR¹, wherein R¹ is define above. Thus, in general, an amino acid-containing compound or derivative thereof of the present invention having a nucleophilic moiety can be represented by formula (I): 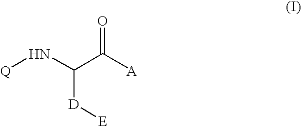
wherein A is a first group that is capable of forming a bond with an amino acid; D is a direct covalent bond or a divalent saturated or unsaturated hydrocarbyl group, or a derivative thereof having from one to ten carbon atoms, preferably from one to five carbon atoms, inclusive; E is a nucleophilic moiety defined above; and Q is a second group that is capable of forming a bond with an amino acid. For example, the first group can be a chain comprising amino acid residues or derivatives thereof. The second group can be another chain comprising amino acid residues or derivatives thereof, —COOR¹, —CONR²R³, —SO₃H, —SO₂NR²R³, or a derivative thereof, wherein R¹ is disclosed above, R² and R³ are independently selected from the group consisting of hydrogen, alkyl, amino protecting groups, chelating moieties, carbohydrates, lipids, and polymer chains. In other embodiments of the present invention, each of A and Q independently can be a chain of plurality of nucleotide residues (“oligonucleotides”) or derivatives thereof that have a terminal moiety capable of forming a bond with an amino acid.

In one embodiment of the present invention, the chain comprising amino acid residues or a derivative thereof having a nucleophilic moiety has a formula (II) or (III): 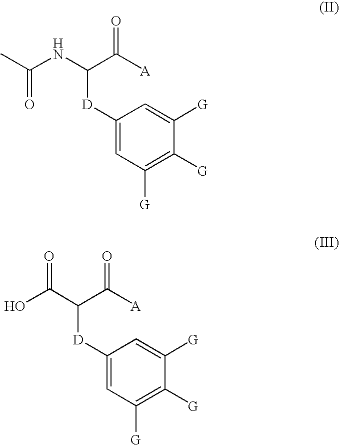
wherein G is an electron-donating group, such as —OR¹, or —SR¹, wherein R¹ is define above.

In a preferred embodiment, the electron-donating group is the methoxy group, the nucleophilic moiety is 3,4,5-trimethoxyphenyl or 3,4,5-trimethoxybenzyl, and D is methylene or ethylene.

Halogenated electrophilic compounds suitable for a labeling reaction with a nucleophilic moiety of a peptide or peptide derivative of the present invention include substituted or unsubstituted N-fluoropyridimium salts, N-fluorobenzene sulfonamide, N-fluoro-N′-chloromethyl-1,4-diaza-bicyclo{2.2.2} octane salts, N-fluoro perfluoro piperidines, substituted or unsubstituted N-chloropyridimium salts, N-chlorobenzene sulfonamide, N-chloro-N′-chloromethyl-1,4-diaza-bicyclo {2.2.2} octane salts, N-chloro perchloro piperidines, substituted or unsubstituted N-bromopyridimium salts, N-bromobenzene sulfonamide, N-bromo-N′-chloromethyl-1,4-diaza-bicyclo{2.2.2} octane salts, and N-bromo perbromo piperidines. In one embodiment, the aforementioned salts are triflate salts or tetrafluoroborate salts.

In particular, halogenated electrophilic compounds suitable for a labeling reaction with a nucleophilic moiety of a peptide or peptide derivative of the present invention have the following formulas: 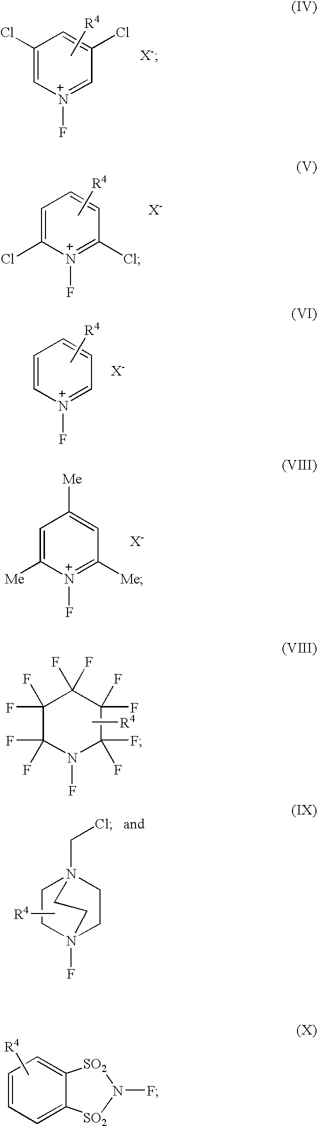
wherein Me is CH₃, X⁻ is —CF₃SO₃⁻ (triflate) or BF₄⁻, and R⁴ is a substituted or unsubstituted alkyl or alkenyl group having up to and including 5 carbon atoms.

In one preferred embodiment, the halogenated electrophilic compound is N-fluoro-2,6-dichloro pyridinium triflate.

### EXAMPLE

### Direct Labeling of Peptide with Halogen

Solid-Phase Peptide Synthesis

First, the solid-phase synthesis technique was employed for the production of two peptide sequences, each having a nucleophilic moiety. The peptides sequences were J₁FLGFL-NH₂ and J₂FLGFL-NH₂, wherein J₁ is 3,4,5-trimethoxybenzyl glycine and J₂ is 3-(3,4,5-trimethoxyphenyl)propionic acid. The syntheses of both sequences were equally successful, although the absence of the amino group in J₂ makes it usable as a terminus only. On the other hand, J₁ could be inserted anywhere along the peptide chain. For these model compounds, the sequence was chosen to be chemically non-reactive and with high organic solubility. The J₂ peptide was synthesized to evaluate relative reactivity in comparison to the J₁ series and to gauge whether J₂ and J₁ moieties could be used within the same peptide sequence.

Peptides were synthesized using standard solid phase techniques with N^{α}-Fmoc-protected amino acids (see; e.g., W. C. Chan and P. D. White (ed.), “Fmoc Solid Phase Peptide Synthesis,” pp. 9-40, Oxford University Press, New York, N.Y. (2000)) using 2,4-dimethoxybenzhydrylamine resin (Rink Amide AM) on a 25 μmole scale (Fmoc=fluorenylmethoxycarbonyl). The peptides were synthesized using a Rainin/Protein Technology Symphony solid phase peptide synthesizer (Woburn, Mass.). Prior to any chemistry, the resin was swelled for one hour in methylene chloride, and subsequently exchanged out with DMF (dimethylformamide) over half-hour or more. Each coupling reaction was carried out at room temperature in DMF with five equivalents of amino acid. Reaction times were typically 45 minutes. The coupling reagent used was HBTU (O-benzotriazolyl-1-yl-N,N,N′,N′-tetramethyluronium hexafluorophosphate), with NMM (N-methylmorpholine) as the base. For each step the coupling agent was delivered at a scale of five equivalents relative to the estimated resin capacity, and reaction carried out in 2.5 ml of 0.4 M NMM solution in DMF.

Following each coupling reaction, the N-terminal Fmoc-protected amine was deprotected by applying 20% piperidine in DMF twice at room temperature for approximately 15 minutes. After the addition of the last residue the resin, still on the peptide synthesizer, was rinsed thoroughly with DMF and methylene chloride.

To couple the J₁ or J₂ to the N-terminus of the peptide, the amino acid, HBTU and NMM was added to the resin in the same manner as the amino acids. The reaction typically proceeded for 3 hours, at the end of which period the Fmoc Group was removed for X₁. After the reaction, the N-terminal amine group of the peptide was capped with an acyl group by adding 0.05 M acetic anhydride, 0.2 M NM and 0.05 M HBTU in a total volume of 2.5 ml DMF. Post reaction, the resin was thoroughly washed with DMF and methylene chloride and dried under a stream of nitrogen.

Peptide Fluorination

A parallel fluorination setup was employed in order to ensure similar reaction conditions for all the individual peptide compositions within each fluorination experiment. This was accomplished using an array of Teflon tubes fritted at the bottom, arranged such that simultaneous addition of a reagent or solvent can be achieved from the top, while simultaneous removal of liquid can be made by applying vacuum below the frit.

Each fluorination tube was loaded with resin beads containing one of the peptide to be fluorinated, amounting to 6 μmol peptide/tube (8.2+/−0.1 mg beads/tube). The tubes were kept in a high vacuum desiccator overnight. Prior to fluorination, the resins were swelled for 1 hr. by adding 0.25 ml dry dichloromethane. A 0.1 M solution of N-fluoro-2,6-dichloropyridinium triflate(40) in dry acetonitrile was prepared prior to use.

The dichloromethane solvent used for swelling was drawn off, replaced with fresh dry solvent (0.25 ml/tube) and to each tube were added 130 μl of the fluorinating solution (1.1 equivalents vs. peptide, corrected for the electron-rich Rink resin linker). Upon a contact time of 15 minutes, the liquids were drawn off, the beads were washed with fresh dichloromethane and the resin was submitted to the cleavage protocol.

Fluorinated Peptide Cleavage from Resin

To cleave the peptides from the resin a cocktail consisting of 1 ml TFA (trifluoacetic acid), 2.5% TIS (triisopropylsilane) and 2.5% water was used. The resin and cocktail were stirred at room temperature for approximately 3 to 4 hours. The resin beads were filtered off using glass wool, followed by rinsing with 2-3 ml of TFA. The peptide was precipitated with 40 ml of ice-cold ether and centrifuged at 3000-4000 rpm until the precipitate formed a pellet at the bottom of the centrifuge tube. The ether was decanted, and the pellet was resuspended in cold ether (40 ml) and centrifuged again; the process was repeated two to three times. During the final wash 10 ml of Millipore water was added to 30 ml of cold ether, and the mixture was centrifuged again. The ether was decanted. The aqueous layer, containing the crude peptide, was transferred to a round bottom flask for lyophilization.

Mass Spectrometry (MALDI-TOF) Characterization

Peptide molecular weight, and hence fluorination, were analyzed using Time of Flight MALDI mass spectrometry in the reflectron mode (Applied Biosystems Voyager-DE STR, Framingham, Mass.).

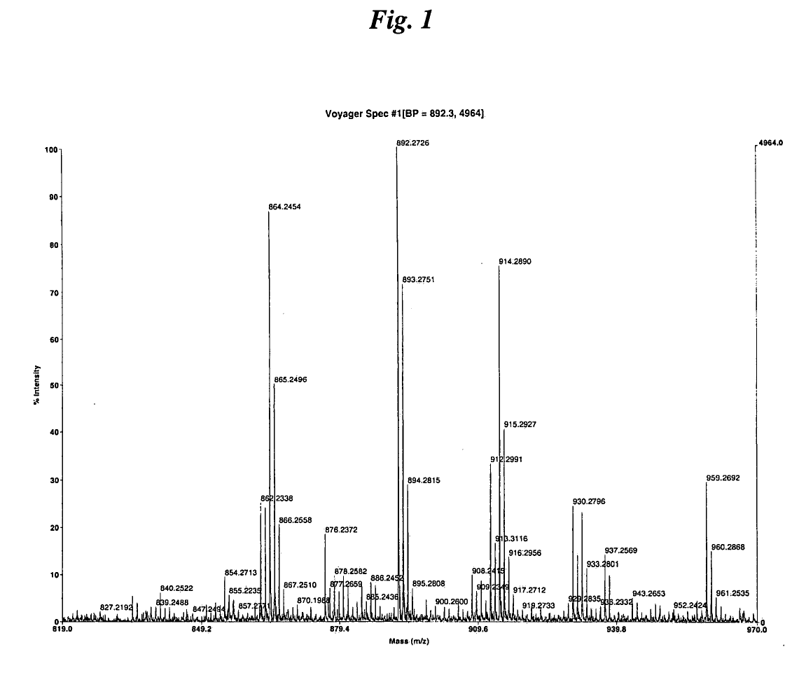

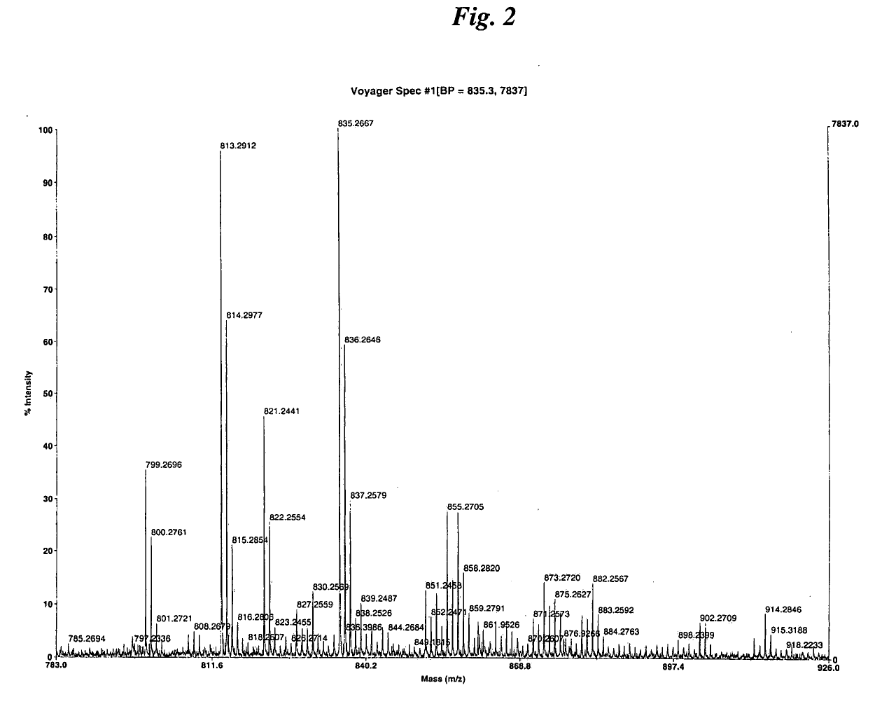

Mass Spectrometry confirmed that both series of compounds, J₁FLGFL-NH₂ and J₂FLGFL-NH₂, were successfully fluorinated. Although the degree of fluorination was not quantified, the predominant peak in the MALDI spectra was that of the fluorinated species. FIG. 1 shows the mass spectrum (MALDI-TOF) of J₁FLGFL-NH₂, wherein J₁ is 3,4,5-trimethoxybenzyl glycine. The expected molecular weight of about 894 is seen in the spectrum. FIG. 2 shows the mass spectrum (MALDI-TOF) of J₂FLGFL-NH₂, wherein J₂ is 3-(3,4,5-trimethoxyphenyl)propionic acid. The expected molecular weight of about 836 is seen in the spectrum.

Carboxyl hypofluorites are another suitable class of halogenated electrophilic compounds when the desired halogen is fluorine. Carboxyl hypofluorites can be generated in-situ by the method described in S. Rozen et al., “Acetyl Hypofluorite, the First Member of a New Family of Organic Compounds,” J.C.S. Chem. Comm., pp. 443-44 (1981). For example, in one non-limiting experiment demonstrating the use of a hypofluorite to fluorinate a peptide, the procedure was as follows.

Resin beads on which a peptide was synthesiezed (at a loading of 0.45 micromoles of peptide/mg of beads) were swollen in fluorotrichloromethane (Freon-11) for 2 hours at 0 C prior to fluorination. To a Teflon vial with cap and a teflon lined septum was added a 9/1 v/v mixture of Freon-11/acetic acid (0.44 ml/micromole of peptide), and the mixture was cooled to −78 C with a dry ice-acetone mixture. A gaseous mixture of 1% (v/v) F₂ in N₂ was bubbled through (1 ml/micromole peptide), and then the slurry was sparged with N₂. A capillary passed through a syringe needle was used both for dilute F₂ addition and for the N₂ sparging. To this mixture was quickly added a suspension of the beads containing the peptide in Freon-1 via canula. The mixture was stirred at −78 C for 5 minutes, then the solvent was drawn off and the beads were washed with dichloromethane. Typically, the reaction was conducted on 7-16 mg of resin-bound peptide (3.15-7.2 micromoles peptide) with the reagent ratios sateted above. Cleavage of the fluorinated peptide was performed as in the example disclosed above, and fluorination was confirmed by MALDI-TOF.

Thus, the method of the present invention can be used rapidly and conveniently to produce a radioactive halide labeled amino acid-containing compound. For example, an amino acid-containing compound can be labeled with a short-lived radioactive halogen, such as ¹⁸F, only a short time before the labeled compound is to be used to avoid a substantial decay of the radioactive level, from which other methods typically suffer.

Similarly, an amino acid-containing compound or a derivative thereof can be labeled with a halogen other than fluorine. Moreover, the procedure of the present invention, as disclosed above, is equally applicable to a radioactive halogen such as any of the radioisotope of fluorine, chlorine, bromine, or iodine disclosed above.

In general, an amino acid-containing compound or a derivative thereof that can be labeled with the method of the present invention comprises at least one amino acid residue in a backbone chain. For example, such an amino acid-containing compound can be a protein or a fragment thereof. Further, such a protein or fragment thereof labeled with a radioactive halogen can be targeted to disease site when such protein preferentially binds a marker substance that is produced by or associated with the diseased tissue. Thus, a labeled protein or fragment thereof produced according to the method of the present invention can serve as a diagnostic imaging or therapeutic agent.

The amino acid-containing compound may also be replaced by a peptide nucleic acid (“PNA”). PNAs are oligomers, the backbone chains of which comprise repeating units of N-(2-aminoethyl)-glycine, wherein the amino nitrogen of the glycine moiety is linked to one of five heterocyclic bases (adenine, guanine, cytosine, thymine, and uracil) through a methyl carbonyl linkage. See; e.g., U.S. Pat. No. 6,395,474.

In still another embodiment, the amino-acid containing compound comprises at least one amino-acid residue and at least a residue of at least another type of monomeric units in the backbone chain, wherein the nucleophilic moiety is conjugated to the at least one amino-acid residue. For example, the at least another type of monomeric units can comprise a chain of plurality of nucleotides (“oligonucleotides”) or derivatives thereof having an amine functional group at a terminus. In one embodiment, the at least one amino-acid residue is attached to the terminal amine group of the chain of nucleotides or derivatives thereof.

In one embodiment, when the radioactive halogen is capable of emitting positrons, the labeled compound is used to image a portion of the body using positron emission tomography (“PET”).

In an embodiment, the proteins of interest in the present invention are antibodies and antibody fragments. The terms “antibodies” and “antibody fragments” mean generally immunoglobulins or fragments thereof that specifically bind to antigens to form immune complexes.

The antibody may be a whole immunoglobulin of any class; e.g., IgG, IgM, IgA, IgD, IgE, chimeric or hybrid antibodies with dual or multiple antigen or epitope specificities. It can be a polyclonal antibody, preferably an affinity-purified antibody from a human. It can be an antibody from an appropriate animal; e.g., a primate, goat, rabbit, mouse, or the like. If the target site-binding region is obtained from a non-human species, it is preferred that the target species is humanized to reduce immunogenicity of the non-human antibodies, for use in human diagnostic or therapeutic applications. Such a humanized antibody or fragment thereof is also termed “chimeric.” For example, a chimeric antibody comprises non-human (such as murine) variable regions and human constant regions. A chimeric antibody fragment can comprise a variable binding sequence or complementarity-determining regions (“CDR”) derived from a non-human antibody within a human variable region framework domain. Monoclonal antibodies are also suitable for use in the present invention, and are preferred because of their high specificities. They are readily prepared by what are now considered conventional procedures of immunization of mammals with an immunogenic antigen preparation, fusion of immune lymph or spleen cells with an immortal myeloma cell line, and isolation of specific hybridoma clones. More unconventional methods of preparing monoclonal antibodies are not excluded, such as interspecies fusions and genetic engineering manipulations of hypervariable regions, since it is primarily the antigen specificity of the antibodies that affects their utility in the present invention. It will be appreciated that newer techniques for production of monoclonal antibodies (“MAb”) can also be used; e.g., human MAbs, interspecies MAbs, chimeric (e.g., human/mouse) MAbs, genetically engineered antibodies, and the like.

Antibody fragments useful in the present invention include F(ab′)₂, F(ab)₂, Fab′, Fab, Fv, and the like including hybrid fragments. Preferred fragments are Fab′, F(ab′)₂, Fab, and F(ab)₂. Also useful are any subfragments retaining the hypervariable, antigen-binding region of an immunoglobulin and having a size similar to or smaller than a Fab′ fragment. An antibody fragment can include genetically engineered and/or recombinant proteins, whether single-chain or multiple-chain, which incorporate an antigen-binding site and otherwise function in vivo as targeting species in substantially the same way as natural immunoglobulin fragments. Such single-chain binding molecules are disclosed in U.S. Pat. No. 4,946,778. Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole immunoglobulin. The fragments may also be produced by genetic engineering.

It should be noted that mixtures of antibodies and immunoglobulin classes can be used, as can hybrid antibodies. Multispecific, including bispecific and hybrid, antibodies and antibody fragments are sometimes desirable in the present invention for detecting and treating lesions and comprise at least two different substantially monospecific antibodies or antibody fragments, wherein at least two of said antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to the anti-tumor marker hybrids disclosed in U.S. Pat. No. 4,361,544. Other techniques for preparing hybrid antibodies are disclosed in; e.g., U.S. Pat. Nos. 4,474,893 and 4,479,895, and in Milstein et al., Immunology Today, Vol. 5, 299 (1984).

In another aspect, the present invention provides a set of separate compounds comprising a first compound comprising an amino acid-containing compound, such as a peptide or peptide derivative, that comprises a nucleophilic moiety, and a second compound that is electrophilic and comprises a halide moiety. The compounds readily react with one another to produce a halide-labeled peptide or peptide derivative, which is produced substantially at the time of use. For example, the first compound may be stored over an extended period of time at the site of future use. The second compound that comprises a radioactive halide moiety is provided shortly before or at the time a radioactive halide-labeled peptide need be produced. Such a set of compounds can constitute a kit for the production of a radioactive diagnostic imaging agent or a radioactive therapeutic pharmaceutical that is the product of the reaction of the compounds.

While various embodiments are described herein, it will be appreciated from the specification that various combinations of elements, variations, equivalents, or improvements therein may be made by those skilled in the art, and are still within the scope of the invention as defined in the appended claims.

What is claimed is:

1. A method for labeling an amino acid-containing compound with a halide moiety, the method comprising reacting a halogenated electrophilic compound with a nucleophilic moiety of the amino acid-containing compound, wherein the nucleophilic moiety comprises at least one aromatic ring that is substituted with at least one electron-donating group, and the halogenated electrophilc compound is selected from the group consisting of substituted and unsubstituted N-fluoropyridimium salts, N-fluorobenzene sulfonamide, N-fluoro-N′-chloromethyl-1,4-diaza-bicyclo{2.2.2} octane salts, N-fluoro perfluoro piperidines, substituted or unsubstituted N-chloropyridimium salts, N-chlorobenzene sulfonamide, N-chloro-N′-chloromethyl-1,4-diaza-bicyclo{2.2.2} octane salts, N-chloro perchloro piperidines, substituted or unsubstituted N-bromopyridimium salts, N-bromobenzene sulfonamide, N-bromo-N′-chloromethyl-1,4-diaza-bicyclo{2.2.2} octane salts, and N-bromo perbromo piperidines.

2. The method according to claim 1, further comprising providing the nucleophilic moiety to the amino acid-containing compound before the step of reacting.

3. The method according to claim 2, wherein said providing the nucleophilic moiety to the amino acid-containing compound comprises linking the nucleophilic moiety to a residue of the amino acid-containing compound.

4. The method according to claim 1, wherein the nucleophilic moiety comprises from one to three aromatic rings which are substituted with at least one electron-donating group selected from the group consisting of —OR¹ and —SR¹, wherein R¹ is —CHₘHₘ₊₁ and 1≦m≦5.

5. The method according to claim 4, wherein the nucleophilic moiety is selected from the group consisting of trimethoxyphenyl and trimethoxybenzyl.

6. The method according to claim 1, wherein the halogenated electrophilc compound is selected from the group consisting of substituted and unsubstituted N-fluoropyridimium salts.

7. The method according to claim 6, wherein the halogenated electrophilc compound is N-fluoro-2,6-dichloro pyridimium triflate.

8. The method according to claim 1, wherein the halogenated electrophilc compound comprises a radioactive halogen.

9. The method according to claim 8, wherein the radioactive halogen is ¹⁸F.

10. The method according to claim 1, wherein the amino acid-containing compound is selected from the group consisting of peptides, peptides derivatives, proteins, antibodies, amino acid-containing oligonucleotides, derivatives thereof, and fragments thereof.

11. A method for producing a labeled amino acid-containing compound, the method comprising:
attaching a group comprising a nucleophilic moiety to an unlabeled amino acid-containing compound; and
reacting a halogenated electrophilic compound with the nucleophilic moiety that is attached to the amino acid-containing compound;
wherein the nucleophilic moiety comprises at least one aromatic ring that is substituted with at least one electron-donating group, and the halogenated electrophilc compound is selected from the group consisting of substituted and unsubstituted N-fluoropyridimium salts, N-fluorobenzene sulfonamide, N-fluoro-N′-chloromethyl-1,4-diaza-bicyclo{2.2.2} octane salts, N-fluoro perfluoro piperidines, substituted or unsubstituted N-chloropyridimium salts, N-chlorobenzene sulfonamide, N-chloro-N′-chloromethyl-1,4-diaza-bicyclo{2.2.2} octane salts, N-chloro perchloro piperidines, substituted or unsubstituted N-bromopyridimium salts, N-bromobenzene sulfonamide, N-bromo-N′-chloromethyl-1,4-diaza-bicyclo{2.2.2} octane salts, and N-bromo perbromo piperidines.

12. The method according to claim 11, wherein said attaching comprising inserting a residue of a derivative of an amino acid in a chain of the amino acid-containing compound, said residue comprising said nucleophilic moiety.

13. A labeled amino acid-containing compound having a formula of 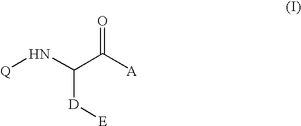
wherein at least one of A and Q is independently selected from the group consisting of peptides, proteins, antibodies, peptide nucleic acids, amino acid-containing oligonucleotides, derivatives thereof, and fragments thereof; D is selected from the group consisting of a direct covalent bond, divalent saturated or unsaturated hydrocarbyl groups, and derivatives thereof having from one to ten carbon atoms, inclusive; and E comprises a nucleophilic moiety.

14. The labeled amino acid-containing compound according to claim 13, wherein at least one of A and Q is a peptide.

15. The labeled amino acid-containing compound according to claim 13, wherein A is a peptide, and Q is selected from the group consisting of peptides, amino acid-containing oligonucleotides, —COOR¹, —CONR²R³, —SO₃H, —SO₂NR²R³, and derivatives thereof; wherein R¹ is —CₘHₘ₊₁, n and m are integers independently selected from the group consisting of 1, 2, 3, 4, and 5; and R² and R³ are independently selected from the group consisting of hydrogen, alkyl, amino protecting groups, chelating moieties, carbohydrates, lipids, and polymer chains.

16. The labeled amino acid-containing compound according to claim 13, wherein D is selected from the group consisting of —(O—CH₂—CH₂)ₙ—, —(S—CH₂—CH₂)ₙ—, or —(NR¹—CH₂-CH₂)ₙ—, R¹ is —CₘHₘ₊₁, and n and m are integers independently selected from the group consisting of 1, 2, 3, 4, and 5.

17. The labeled amino acid-containing compound according to claim 13, wherein E comprises from one to three aromatic rings, substituted with at least one electron-donating group selected from the group consisting of —OR¹ and —SR¹, wherein R¹ is —CₘHₘ₊₁ and m is an integer such that 1≦m≦5.

18. The labeled amino acid-containing compound according to claim 17, wherein E is selected from the group consisting of trimethoxyphenyl and trimethoxybenzyl.

19. The labeled amino acid-containing compound according to claim 13, wherein the amino acid-containing compound is labeled with a halide moiety.

20. The labeled amino acid-containing compound according to claim 19, wherein the halide moiety is radioactive.

21. The labeled amino acid-containing compound according to claim 20, wherein the radioactive halide moiety emits positrons.

22. The labeled amino acid-containing compound according to claim 20, wherein the halide moiety is ¹⁸F.

23. A kit comprising a first compound and a second compound that are kept separate, wherein said first compound comprises an amino acid-containing compound comprising a nucleophilic moiety that comprises at least one aromatic ring that is substituted with at least one electron-donating group, and said second compound is an halogenated electrophilic compound that is selected from the group consisting of substituted and unsubstituted N-fluoropyridimium salts, N-fluorobenzene sulfonamide, N-fluoro-N′-chloromethyl-1,4-diaza-bicyclo{2.2.2} octane salts, N-fluoro perfluoro piperidines, substituted or unsubstituted N-chloropyridimium salts, N-chlorobenzene sulfonamide, N-chloro-N′-chloromethyl-1,4-diaza-bicyclo {2.2.2} octane salts, N-chloro perchloro piperidines, substituted or unsubstituted N-bromopyridimium salts, N-bromobenzene sulfonamide, N-bromo-N′-chloromethyl-1,4-diaza-bicyclo{2.2.2} octane salts, and N-bromo perbromo piperidines.

24. The kit according to claim 23, wherein the nucleophilic moiety comprises from one to three aromatic rings which are substituted with at least one electron-donating group selected from the group consisting of —OR¹ and SR¹, wherein R¹ is —HₘHₘ₊₁ and 1≦m≦5.

25. The kit according to claim 24, wherein the nucleophilic moiety is selected from the group consisting of trimethoxyphenyl and trimethoxybenzyl.

26. The kit according to claim 23, wherein the halogenated electrophilc compound is selected from the group consisting of substituted and unsubstituted N-fluoropyridimium salts.

27. The kit according to claim 26, wherein the halogenated electrophilc compound is N-fluoro-2,6-dichloro pyridimium triflate.

28. The kit according to claim 23, wherein the halogenated electrophilc compound comprises a radioactive halogen.

29. The kit according to claim 28, wherein the radioactive halogen is ¹⁸F.

30. The kit according to claim 23, wherein at least a portion of the amino acid-containing compound is selected from the group consisting of peptides, peptides derivatives, peptide nucleic acids, proteins, antibodies, amino acid-containing oligonucleotides, derivatives thereof, and fragments thereof.
